(19) United States
(12) Patent Application Publication
(10) Pub. No.: US 2004/0146556 A1
(43) Pub. Date: Jul. 29, 2004

(54) Oral extended release tablets and methods of making and using the same

(76) Inventors: Robert M. Noack, Grand Rapids, MI (US); John M. Heimlich, Portage, MI (US); Ernest John Lee, Kalamazoo, MI (US)

Correspondence Address: Karen B. King, Pharmacia Corporation, P. O. Box 1027, St. Louis, MO, 63006, US

(21) Appl. No.: 10/696,044

(22) Filed: Oct. 29, 2003

Related U.S. Application Data
(60) Provisional application No. 60/422,418, filed on Oct. 30, 2002.

(51) Int. Cl. A61K 9/22

(52) U.S. Cl. 424/468

(57) ABSTRACT

The present invention is directed to oral dosage forms for extended release, including a dosage form for pH independent extended release, of at least one drug to a subject. The present invention is also directed to methods of making and using the dosage forms to treat or prevent a subject for various conditions. Specific extended release formulations of crystalline clindamycin free base are also provided. The crystalline clindamycin free base oral formulations of the present invention provide a means for treating or preventing gram-positive bacterial infections with a minimal number of treatments per day, potentially, as little as once or twice per day.

### CROSS-REFERENCE TO RELATED APPLICATIONS

This application claims the benefit of U.S. Provisional Application No. 60/422,418, filed Oct. 30,2002.

### FIELD OF THE INVENTION

The present invention relates to extended release tablet dosage forms, including, forms that release at least one drug contained therein independently of changes in pH. The present invention particularly relates to extended release tablet dosage forms that release at least one drug contained therein at a controlled rate of release as the tablet passes from the highly acidic gastric juices into the higher pH environment of the lower gastrointestinal tract of a subject, after oral administration thereto.

### BACKGROUND

The solubility characteristics of some drugs or other pharmaceutically active agents vary little with changes in pH, while others vary considerably under the same or similar changing pH conditions. Drugs that are more soluble at low pH ranges and less soluble at higher pH ranges tend to be released quickly in the acidic environment of the upper gastrointestinal tract, after oral administration, before the drug reaches the intestine. The rate of release of such drugs slows down after the drug leaves the stomach and enters the intestine, where the pH is higher. However, by then, so much drug has been released that only a small portion of the drug remains. Without any mechanism to slow drug release, all of any such drug is released within a few short hours. In many cases, many doses of the same drug must be taken throughout a day to maintain a therapeutic level of the drug in a subject. When the drug is an antibiotic, pH independent extended release is desirable in order to minimize the number of doses any given subject need take in a given day to treat or prevent an illness or infection, and to increase the likelihood of compliance with a treatment regimen.

Extended release dosage forms have been developed that extend the rate at which a drug is released from the dosage form. Most such dosage forms control the rate of release through a coating of a core containing the drug, while others control the rate of release through a controlled release system within the core of the dosage form. Some of the extended release means are pH dependent, while others are not. Below is a summary of some of the many known means of extended release from oral dosage forms.

Some formulations are provided in the form of a tablet comprising a polymeric matrix with a drug distributed through the matrix. The matrix is designed to control the rate of delivery of the drug, after administration to a subject. At least some of the drug in any such system is present at or sufficiently close to the outer surface, and tends to be released from the tablet a considerably faster rate than drug contained closer to the interior of the matrix. This effect is commonly referred to as the “burst effect”. The effect can be, but is not necessarily related to changes in the pH of the environment of such a tablet.

Some formulations comprise a core containing a drug and a coating of a swellable polymer coating that swells in an aqueous environment, enabling the drug to diffuse through the stagnant liquid phase contained in the polymer. Such formulations can be sensitive to pH.

EP 0 572 942 B2 (MONSANTO ITALIA) discloses a variation of the diffusion model. The tablet comprises a tablet core containing a drug and excipients, an intermediate layer with a hydrophilic swellable polymer or a copolymer or mix, and a coating whose dissolution activates the process of the intermediate layer swelling, dissolving, or eroding. When the intermediate layer swells, it delays the release of the drug for a specific amount of time, independent of pH. The core can be in the form of a matrix, although the nature of the possible matrix configuration is not disclosed. This tablet appears to be designed to delay, rather than to extend release of the drug, possibly until after the tablet passes through the acidic environment of the stomach into the lower gastrointestinal tract.

WO 98/03161 (DEXCEL, LTD) discloses another variation of the diffusion model, a controlled release tablet comprising a controlled release core composition of a drug incorporated into a polymeric carrier and diffusible therefrom at a predetermined rate, and a coating comprising a water insoluble and water impermeable polymeric material having at least one channeling agent dispersed in the coating. The channeling agent is soluble upon contact with the medium into which the drug is to be released. Since the coating material, which is insoluble in water, becomes porous due to solubilization of the channeling agent, it appears the core is in constant contact with the external environment. Drug release would, therefore, depend upon the solubility of the drug in the medium outside the tablet, a medium that changes pH as the tablet passes through the gastrointestinal tract.

Other formulations comprise a core containing a drug, and a coating covering the core that disintegrates by a process that is dependent upon particular environmental conditions, such as changes in pH or the presence of certain enzymes, leaving the core exposed to rapid dissolution after the coating disintegrates. U.S. Pat. No. 6,74,669 discloses a tablet with a core of a hydrophilic polymer, a drug, and excipients, and an enteric coat covering the outer surface of the tablet. The enteric coat protects the core from exposure to the external environment until after the tablet passes out of the highly acidic environment of the stomach and into the higher pH environment of the intestines, at which time the enteric coat dissolves, and the drug is rapidly released from the exposed core.

U.S. Pat. No. 6,068,856 (Sachs et al.) discloses an enteric coated tablet similar to the '669 patent, described above, except that the tablet core comprises film-forming polymers and water-soluble pore formers. Otherwise, a drug is released from the tablet disclosed in the '856 patent as it is from the '669 patent.

Other dosage forms use a matrix in a coated tablet core to control drug release rates. U.S. Pat. No. 6,068,859 (Curatoto et al.) discloses a tablet comprising beads of azithromycin are dispersed in a matrix that retards release of azithromycin into the lumenal fluid of the GI tract. The tablet coating can comprise a hydrophilic polymer, such as HPMC, or an impermeable coating with an orifice. The '859 patent also discloses that, alternatively, the beads can be coated with a film, and the tablet can use osmotic pressure for delivery of the azithromycin.

The alternative azithromycin osmotic delivery system of the '859 patent, cited above, is only one of many osmotic systems developed for drug delivery. For examples of such systems, see U.S. Pat. Nos. 4,880,631; 5,458,887; and 4,096,238. Osmotic systems require the use of a mechanism whereby osmotic pressure can be increased when release is desired, and a means for controlling that release, such as a membrane with controlled porosity. Some osmotic systems, such as the tablets disclosed in U.S. Pat. No. 4,687,660, include osmotic enhancing agents. Other osmotic systems, such as the tablets disclosed in U.S. Pat. Nos. 4,994,273 and 4,946,686, include at least one solubility modulating agent that can exert an effect on the water solubility of the drug being delivered from the device without chemical modification of the drug.

Known extended release dosage forms, such as those described above, either fail to provide pH independent drug release of pH sensitive drugs, or they provide a system for such release that is so complex as to make production of the dosage forms cost prohibitive. Some known extended release dosage forms such as uncoated matrix release forms also exhibit a “burst effect” that may or may not be related to changes in pH. (See paragraph 0004, above, for a description of the burst effect.) In one aspect, what is needed is an extended release dosage form that eliminates or substantially controls the burst effect. In another aspect, what is needed is a new oral tablet dosage form that provides a pH independent sustained release dosage form, preferably one with a zero-order release profile. As is illustrated below, embodiments of the oral tablet dosage forms of the present invention meet each of these needs.

### BRIEF SUMMARY OF THE INVENTION

The dosage form of the present invention uses a tablet core containing at least one drug with its own controlled release mechanism, wherein the outer surface of the core is covered by an enteric coating comprising a pore former distributed therein.

In one embodiment of the invention, the tablet core is a matrix comprising a polymer and a drug distributed therein, and the enteric coating comprising a pore former minimizes any burst effect otherwise associated with the matrix.

In another embodiment of the present invention, the dosage form has a capacity to release the drug contained therein at an essentially constant rate of release, even through the dramatic changes in pH that occur as the dosage form passes from the stomach to the lower gastrointestinal tract of a subject, after oral administration.

The dosage forms of the present invention can provide vehicles for the administration of any one of a number of different drugs to a subject, including antibiotics. Some drugs have solubility characteristics, such as being more soluble in an acidic environment and less soluble in a basic environment, that make them particularly well suited for delivery using the pH independent release dosage form of the present invention. In one embodiment of that dosage form, the drug in the tablet core is crystalline clindamycin free base, a drug having such pH dependent solubility characteristics.

The extended release dosage forms of the present invention utilize a combination of at least two different mechanisms to extend the rate of release of the drug therefrom. The pore formers in the enteric coating provide the first such mechanism. The enteric coating is designed to remain intact in the upper gastrointestinal tract, including in the highly acidic environment of the stomach; while, pore-formers in the enteric coating allow a limited amount of drug from the tablet core to be released into the upper gastrointestinal tract. When the extended release dosage form passes from the stomach into the lower gastrointestinal tract (e.g. the large and small intestines), the pH of the environment surrounding the tablet rises, and the enteric coating dissolves. At that point, the remaining tablet core is designed to release the remaining drug contained therein at an extended rate of release. When the dosage form is the pH independent release dosage form of the present invention, the overall rate of release, from oral administration through the stomach and most of the lower gastrointestinal tract is preferably substantially constant, more preferably a zero-order rate of release.

In another embodiment, the present invention relates to a method of treating or preventing a gram positive infection by oral administration of either extended release crystalline clindamycin free base dosage form, described above, preferably the pH independent extended release crystalline clindamycin free base dosage form, described immediately above.

In another embodiment, the invention is a method of making a pH independent extended release dosage form of the present invention.

### BRIEF DESCRIPTION OF THE DRAWING(S)

FIG. 1 is a plot of in vitro drug release data from four dosage forms with tablet cores of crystalline clindamycin free base, hydroxypropyl methylcelulose (hereinafter, “HPMC”), magnesium stearate, and a buffer, three of which sets of tablet cores were coated with an enteric coating containing HPMC as a pore-former (Formulations 1 through 3), and one set of which cores was uncoated (Formulation 4).

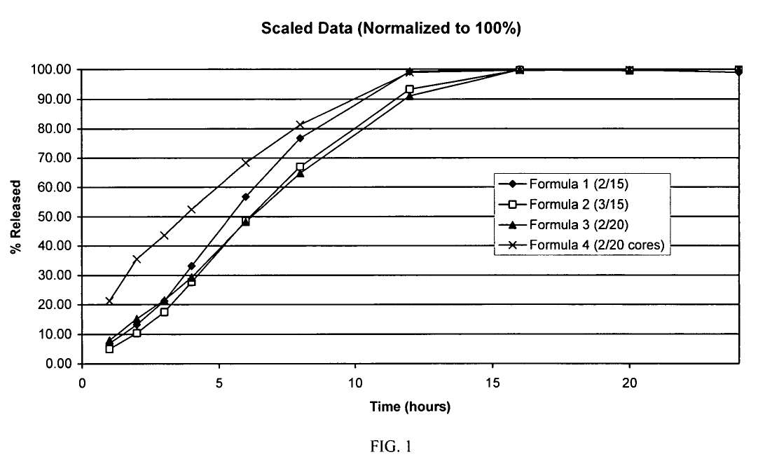

FIG. 2 is a plot of in vitro drug release data from four dosage forms with tablet cores of the same type as described in FIG. 1, above, with the following exceptions: Tablets of Formula 5 did not include a buffer, nor were they coated. Tablets of Formula 6 did not include a buffer, but were coated with an enteric coating, with no pore former. Formula 7 included a buffer, but no coating. Formula 8 included a buffer and a coating with a pore former, as described in FIG. 1, above. Tablets of Formula 9 included a buffer and were uncoated.

FIG. 3 is a plot of drug release data from uncoated (Formulation 14) and enteric/HPMC coated (Formulation 15) unbuffered tablet cores containing crystalline clindamycin free base and HPMC, after storage under various conditions.

FIG. 4 is a plot of in vitro drug release data from uncoated matrices of five different concentrations of one particular HPMC polymer and crystalline clindamycin free base (Formulae 16-20).

FIG. 5 is a plot of in vitro drug release data from three enteric/HPMC coated formulations of crystalline clindamycin free base, with cores containing varying amounts of NaCMC (Formulae 21-22).

### DETAILED DESCRIPTION OF THE INVENTION

As used herein, the term “pH independent release” refers to a rate of release of a drug from a dosage form that does not change when the pH of the environment in which the dosage form is found is changed from an acidic pH to a higher pH.

As used herein, the term “zero-order release” refers to a uniform or nearly uniform rate of release of a drug from a dosage form during a given period of release, a rate of release that is independent of the concentration of drug in the dosage form. A dosage form with a zero-order release profile is referred to herein as a “zero-order dosage form.” Any zero-order dosage form has the advantage of providing maximum therapeutic value while minimizing side effects.

The term “oral administration,” as used herein, refers a form of delivery of a dosage form of a drug to a subject, wherein the dosage form is placed in the mouth of the subject and swallowed.

The term “orally deliverable” herein means suitable for oral administration.

The term “dose unit” herein means a portion of a pharmaceutical composition that contains an amount of a therapeutic agent suitable for a single oral administration to provide a therapeutic effect. Typically, one dose unit, or a small plurality (up to about 4) of dose units, administered as a single oral administration, provides a sufficient amount of the agent to result in the desired effect.

The term “enteric coating”, as used herein, refers to a tablet coating that is resistant to gastric juice, and which dissolves after a dosage form with the enteric coating passes out of the stomach, after oral administration to a subject.

The term “excipient”, as used herein, means any substance, not itself a therapeutic agent, used as a carrier or vehicle for delivery of a therapeutic agent to a subject or added to a pharmaceutical composition to improve its handling, storage, disintegration, dispersion, dissolution, release or organoleptic properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, lubricants, glidants, substances added to mask or counteract a disagreeable taste or odor, flavors, dyes, fragrances, and substances added to improve appearance of the composition.

The term “substantially homogeneous” with reference to a pharmaceutical composition that comprises several components means that the components are sufficiently mixed such that individual components are not present as discrete layers and do not form concentration gradients within the composition.

The pH independent extended release characteristics of one embodiment of the dosage form of the present invention result from the combination of an enteric coating with at least one pore former, that allows a limited amount of environmental fluids to reach the tablet core in the upper gastrointestinal tract, thereby permitting a limited amount of drug to be released into the subject at that stage after oral administration thereto. Once the dosage form leaves the highly acidic environment of the stomach and enters the higher pH of the lower gastrointestinal tract, the enteric coating dissolves, and the tablet core matrix controls the rate of release of drug remaining therein. The enteric coating preferably dissolves at a pH of at least about 5.

In addition to a pH independent release rate, the dosage form of the present invention described above preferably has a controlled release rate, more preferably, a zero-order release rate through changes in pH, such as occur when the dosage form passes from the stomach to the upper intestines of a subject after oral administration thereto. In the case of a human being the average pH of the fluids in a stomach is about pH 1. 1, while the average pH of the upper intestinal tract is about pH 5 to about 7.

In another embodiment, the enteric coating with pore former is used to reduce the burst effect associated with matrix tablets. This effect is thought to be related to the size of the surface area of a tablet, and to be caused by the amount of drug located on or near the surface of the tablet. This effect can be minimized through the coating of a tablet core matrix with an enteric coating with pore-former distributed therein, as described above. For this embodiment of the invention, the solubility of the drug in the tablet core need be pH dependent. It is contemplated that any drug could be used in this embodiment of the invention, provided its solubility characteristics allow for containment within the matrix and release therefrom. The enteric coating with pore former effectively minimizes the surface area of the tablet that is initially exposed to solution in the GI tract and thus limits the amount of drug that is initially released. The coating composition, ratio of enteric to pore-former, could be changed to dictate how much the burst is minimized and therefore the release rate of the drug. The coating dissolves when the tablet enters the intestine and the core will take over the control of the tablet release.

The dosage form of the present invention preferably extend the period of drug release compared to uncoated tablet cores having the same composition as the tablet cores of the present dosage forms. The drug in the coated tablet cores of the present invention preferably continue to release the drug into a subject to at least 10 hours, more preferably to at least 12 hours, even more preferably to at least 14 hours, and most preferably to at least 16 hours after oral administration.

The tablet core comprises a matrix of substantially homogeneous components, including a drug and at least one hydrophilic polymer. The components of the tablet core are dry mixed and compressed into tablets. No specialized geometry of the matrix core is necessary in the present invention. The matrix core may be in any shape known in the pharmaceutical industry and suitable for drug delivery, such as in spherical, cylindrical, or conical shape. In the case of cylindrical shape, it generally has flat, convex, or concave surfaces.

The drug in the tablet core diffuses out of the tablet and into the environment surrounding the tablet through channels formed initially through pore forming agents in the enteric coating, and later, after the enteric coating has dissolved, through channels formed in the matrix itself. The tablet core is prepared by conventional dry granulation methods without using a solvent. The enteric coating is applied using a conventional process known in the art. The coated tablets of the present invention have a dual advantage in allowing ease of manufacture and affording medicament release in a substantially linear fashion over an extended period of time.

The tablet core of the dosage form of the present invention comprises a matrix of a drug and a water soluble polymer. Once the tablet exits the highly acidic environment of the stomach and enters the intestine, the coating dissolves therefrom, and the core continues to release drug in a controlled fashion. The controlled release rate of drug from the tablet core, in the absence of a coating can be maintained at the pH of the small and large intestine.

The tablet core comprises at least one hydrophilic polymer. Suitable hydroxypropyl methylcellulose (hereinafter, “HPMC”), hydroxypropylcellulose, or other water soluble or swellable polymers such as sodium carboxymethyl cellulose, xanthan gum, acacia, tragacanth gum, guar gum, karaya gum, alginates, gelatin, and albumin. The hydrophilic polymers are preferably present in amounts ranging from about 5% to about 95%, more preferably from about 10% to about 50% by weight of the system.

The preferred hydrophilic polymers are selected from the group consisting of cellulose ethers, such as hydroxypropylmethylcellulose, hydroxypropylcellulose, methylcellulose, and mixtures thereof. The most preferred hydrophilic polymer is hydroxypropylmethylcellulose (hereinafter referred to as “HPMC”).

The drug in the tablet core is preferably more soluble in an acidic environment and less soluble in an environment with closer to a neutral or with a basic pH. Examples of drugs suitable for inclusion as at least one drug in the tablet core of the present dosage form include, but are not limited to antihistamines, antibiotics, antituberculosis agents, cholinergic agents, antimuscarinics, sympathomimetics, sympatholytic agents, autonomic drugs, iron preparations, haemostatics, cardiac drugs, antihypertensive agents, vasodilators, non-steroidal antiinflammatory agents, opiate agonists, anticonvulsants, tranquilizers, stimulants, barbiturates, sedatives, expectorants, antiemetics, gastrointestinal drugs, heavy metal antagonists, antithyroid agents, genitourinary smooth muscle relaxants and vitamins. To be suitable for use in the dosage forms of the present invention, a drug is preferably provided in a form that is ionizable at a pH at or below pH 5.

Where a given salt form of a drug is too soluble to provide desired extended release characteristics using a dosage form of the present invention, it may be preferred to use a less soluble form, such as a crystalline form, of the same drug in the dosage form. When the drug is clindamycin, the clindamycin can be present as a salt of clindamycin, such as clindamycin hydrochloride or clindamycin phosphate, or as a pharmaceutically active analog of clindamycin, such as analogs disclosed in U.S. Pat. Nos. 3,496,163; 4,568,741; and 3,583,972, incorporated herein by reference. When the antibiotic is clindamycin, the clindamycin is most preferably present as crystalline clindamycin free base. Crystalline clindamycin free base is less soluble than the highly soluble salts and analogs of clindamycin, making its release from the tablet core matrix easier to control than its more soluble counterparts.

Crystalline clindamycin free base is disclosed in U.S. patent application No. 10/228,356, incorporated herein by reference. Crystalline clindamycin free base can be produced by either of the two alternative processes, illustrated in the above-referenced patent application. One illustrative process of preparing crystalline clindamycin free base involves forming the amorphous free base as a precipitate in aqueous medium followed by agitation to crystallize the free base from the precipitate. An illustrative example of the method involves first dissolving a salt of clindamycin, e.g., clindamycin hydrochloride in a solvent, preferably a polar solvent such as, for example, water. This if followed by adding an alkali material, i.e. a base, in an aqueous vehicle such as for example, a NaOH solution, such as, for example, preferably from about 0.01 to about 10 N NaOH solution, more preferably from about 0.1 to about 1 N NaOH, and more preferably about 0.5 N NaOH. This results in precipitation of the amorphous free base. The amorphous free base is then crystallized by agitation of the precipitate by, for example, by sonicating or manually shaking the precipitate, or by both sonicating and manually shaking the precipitate suspended in the aqueous medium. The crystallized free base is then preferably harvested by centrifugation, followed by removal of the liquid portion. The crystallized free base is preferably washed in at least one washing step involving adding a wash solution, sonicating, shaking. centrifuging and removing the wash solution from the crystalline material. The wash solution is preferably aqueous, more preferably water.

In an alternate method, crystalline clindamycin free base can be produced by a slow addition of a clindamycin salt, such as clindamycin hydrochloride, dissolved in a polar solvent such as water to an aqueous alkaline solution containing a water-soluble organic substance, preferably an alcohol co-solvent. The aqueous solution containing an alkali with an alcohol co-solvent is prepared by adding the alkali, i.e. base, in an aqueous vehicle such as, for example, a NaOH solution. The NaOH solution can be, for example, preferably from about 0.01 to about 10 N NaOH solution, more preferably from about 0.1 to about 1 N NaOH, and more preferably about 0.5 N NaOH. The alcohol co-solvent is present, preferably in an amount of from about 2% to about 20%, more preferably from about 5% to about 10%. Any of a number of alcohols that are readily miscible with water can be used, preferably, methanol, ethanol, n-propanol, t-butanol and the like. Typically alcohols of higher molecular weight are less soluble in water and less preferred. Diols such as 1,2, ethanediol (ethylene glycol), 1,2 propanediol (propylene glycol) and 1,2 butanediol and triols such as 1,2,3 propanediol (glycerol) and the like can also be used as co-solvent. It is also possible to use an aqueous solution of a water-soluble organic substance such as, for example, sodium acetate.

An aqueous solution of a clindamycin salt, such as, for example clindamycin hydrochloride is prepared and slowly added to the alkali solution with alcohol co-solvent, preferably over a period of from about 15 minutes to about 4 hours, more preferably from about 30 minutes to about 2 hours and most preferably from about 45 minutes to 75 minutes. Crystallization is allowed to proceed for 1 to 24 hours and the crystalline free base material is isolated by filtration, centrifugation and decanting or the like. In a preferred variation of this method, the clindamycin hydrochloride solution is added in a multi-phase infusion schedule such as, for example, a first phase of slow infusion over about one hour, followed by a faster infusion phase over about 30 min and concluding with slow infusion phase over about one hour.

The material obtained by either of the methods above is isolated and dried, for example, under a stream of humidified nitrogen. The dry material can be further processed such as by grinding to produce a dry powder.

The tablet core of the present dosage form preferably contains a therapeutic amount of the drug. How much of any given drug constitutes a therapeutic amount for a given subject is dependent inter alia on the body weight of the subject. Where the drug is clindamycin, and the subject is a child or a small animal (e.g., a dog), for example, an amount of clindamycin relatively low in the preferred range of about 24 mg/kg/day to about 80 mg/kg/day. An especially preferred amount of clindamycin crystalline free base per dosage form is typically about 24 mg/kg/day to about 64 mg/kg/day, which is likely to provide blood serum concentrations consistent with therapeutic effectiveness. Where the subject is an adult human or a large animal (e.g., a horse), achievement of such blood serum concentrations of clindamycin or of another drug are likely to require dose units containing a relatively greater amount of the drug. For an adult human, a therapeutically effective amount of crystalline clindamycin free base per dosage form in a composition of the present invention is suitably about 500 mg to about 2000 mg, more preferably about 600 mg to about 1800 mg. An especially preferred amount of crystalline clindamycin free base per dosage form for an adult human is about 600 mg to about 1200 mg.

The amount of drug in a given dosage form can be selected to accommodate the desired frequency of administration used to achieve a specified daily dosage. The amount of the unit dosage form of the composition that is administered and the dosage regimen for treating the condition or disorder will depend on a variety of factors, including the age, weight, sex and medical condition of the subject, the severity of the condition or disorder, the route and frequency of administration, and the particular drug selected, and thus may vary widely. One or more dosage forms can be administered up to about 6 times a day. However, the dosage forms of the present invention release at an extended rate, making it possible to provide the desired therapeutic efficacy by administration once-a-day or twice-a-day.

The tablet core of the dosage form of the present invention is coated with an enteric coating comprising an enteric polymer and a pore-former distributed within the enteric polymer. Enteric polymers suitable for use in the present invention include, but are not limited to polyacrylate copolymers such as methacrylic acid/methacrylic acid ester copolymers or methacrylic acid/acrylic acid ester copolymers, such as USP/NF, Types A, B, or C, which are available from Rohm GmbH under the brand name Eudragit™; cellulose derivatives, such as cellulose acetate phthalate, hydroxypropyl methylcellulose pthalate, hydroxypropyl methyl cellulose acetate succinate, and cellulose acetate trimellitate; and polyvinyl acetate phthalate, such as is available from Colorcon, under the brand name SURETERIC®, and the like. In a preferred embodiment of this invention, the enteric polymer is a polyvinyl acetate phtalate.

Suitable water soluble pore-forming agents for use in the enteric coating in the dosage forms of the present invention include, but are not limited to, povidone K 30, polyvinyl alcohol, cellulose derivatives such as hydroxypropyl cellulose, hydroxypropyl methyl cellulose, methyl cellulose or sodium carboxymethylcellulose; sucrose; xylitol, sorbitol, mannitol, maltose, xylose, glucose, potassium chloride, sodium chloride, polysorbate 80, polyethylene glycol, propylene glycol, sodium citrate, or combinations of any of the above. The pore-forming agent preferably comprises hydroxypropyl methyl cellulose.

The composition of the enteric coating is preferably designed to ensure adherence of the coating to the tablet core. Methods for selection of coating compositions that adhere to compressed tablets are known. See, for example, Pharmaceutical Dosage Forms: Tablets, 2^{nd} ed., vol. 1, Lieberman et al., ed. (Marcel Dekker, Inc.; New York, N.Y.; 1989), pp. 266-271, incorporated herein by reference. Additionally, the cores can be subcoated prior to coating with an enteric coating. The subcoat can function; to provide insure that pores in the core are filled in prior to coating with an enteric coat. (insure against coating failure). The sub coat can consist of any film forming formulation examples include Opadry (Colorcon), Opadry II (Colorcon), AMT (Colorcon) and HPMC.

The enteric coating, including the enteric polymer and the pore-forming agent, is preferably about 3% to about 10% by weight of the dosage form of the present invention, with about 4% to about 5% being a more preferred range.

The tablet core or enteric coating or both the tablet core and enteric coating optionally include at least one excipient. Non-limiting examples of excipients suitable for use in the dosage forms of the present invention follow.

Dosage forms of the present invention optionally comprise a buffer, preferably incorporated into the tablet core. When a buffer is present, it is preferably a buffer designed to maintain the pH at a pH range wherein the drug, contained therein, is stable. When the drug is crystalline clindamycin free base, the buffer is preferably potassium phosphate monobasic Other suitable buffers include, but are not limited to potassium citrate, sodium citrate, sodium phosphate dibasic, diethanolamine, monoethanolamine, sodium bicarbonate, TRIS, and THAM. Depending upon the amount of buffer required to stabilize a given drug, and depending upon the size of the tablet core without the buffer, inclusion of a buffer in the tablet core could produce a dosage form that is too large to be orally administrable to a given subject. Surprisingly, the dosage form of the present invention provides sufficient protection for the drug in the matrix core that inclusion of a buffer in the tablet core is not necessary for effective delivery of the drug. See the Examples, below, for an illustration of the stability and pH independent release capacity of one particular drug, crystalline clindamycin free base, from dosage forms of the present invention with and without buffer (Examples 2 through 5).

Dosage forms of the invention, preferably the tablet core matrix, optionally comprise one or more pharmaceutically acceptable diluents as excipients. Suitable diluents illustratively include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of α- and amorphous cellulose (e.g., Rexcel™) and powdered cellulose; calcium carbonate; glycine; bentonite; polyvinylpyrrolidone; and the like. Such diluents, if present, constitute in total about 5% to about 99%, preferably about 10% to about 85%, and more preferably about 10% to about 80%, of the total weight of the composition. The diluent or diluents selected preferably exhibit suitable flow properties and, where tablets are desired, compressibility.

Compositions of the invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose, microcrystalline cellulose, and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; hydroxypropylmethylcellulose; hydroxypropylcellulose (e.g., Klucel™); and ethylcellulose (e.g., Ethocel™). Such binding agents and/or adhesives, if present, constitute in total about 0.5% to about 25%, preferably about 0.75% to about 15%, and more preferably about 1% to about 10%, of the total weight of the composition.

When the drug is clindamycin, microcrystalline cellulose is a particularly preferred binder, because of its known chemical compatibility with that particular drug. The use of extragranular microcrystalline cellulose (that is, microcrystalline cellulose added to a wet granulated composition after a drying step) can also be used to improve hardness (for tablets) and/or disintegration time. Microcrystalline cellulose included in dry granulation similarly improves hardness of a tablet core.

Compositions of the invention optionally comprise one or more pharmaceutically acceptable lubricants (including anti-adherents and/or glidants) as excipients. Suitable lubricants include, either individually or in combination, glyceryl behenate (e.g., Compritol™ 888); stearic acid and salts thereof, including magnesium, calcium and sodium stearates; hydrogenated vegetable oils (e.g., Sterolex™); colloidal silica; talc; waxes; boric acid; sodium benzoate; sodium acetate; sodium fumarate; sodium chloride; DL-leucine; PEG (e.g., Carbowax™ 4000 and Carbowax™ 6000); sodium oleate; sodium lauryl sulfate; and magnesium lauryl sulfate. Such lubricants, if present, constitute in total about 0.1% to about 10%, preferably about 0.2% to about 8%, and more preferably about 0.25% to about 5%, of the total weight of the composition.

Magnesium stearate is a preferred lubricant used, for example, to reduce friction between the equipment and granulated mixture during compression of tablet formulations.

Suitable anti-adherents include talc, cornstarch, DL-leucine, sodium lauryl sulfate and metallic stearates. Talc is a preferred anti-adherent or glidant used, for example, to reduce formulation sticking to equipment surfaces and also to reduce static in the blend. Talc, if present, constitutes about 0.1% to about 10%, more preferably about 0.25% to about 5%, and still more preferably about 0.5% to about 2%, of the total weight of the composition.

Other excipients such as colorants, flavors and sweeteners are known in the pharmaceutical art and can be used in compositions of the present invention.

In one embodiment of the invention, the dosage form comprises: a tablet core comprising an drug and in a water soluble polymer matrix; and an enteric coating comprising an enteric polymer and a pore-former; wherein, the tablet core or the enteric coating or both include at least one excipient. The dosage form comprises at least one excipient preferably selected from the group consisting of pharmaceutically acceptable diluents, binding agents and lubricants. More preferably, the dosage form comprises at least one excipient selected from the group consisting of lactose (most preferably lactose monohydrate), polyvinylpyrrolidone, magnesium stearate and microcrystalline cellulose. Still more preferably, the tablet core of the present dosage form of the present invention comprises microcrystalline cellulose and magnesium stearate.

Standard methods of production are suitably used to produce the dosage forms of the present invention. Dry mixing of intragranular ingredients, followed by granulation, and dry mixing of intragranular ingredients with extragranular ingredients are standard techniques used in the industry. See, for example, Chapter 4 (“Compressed Tablets by Direct Compression,” by Ralph F. Shangraw) of Pharmaceutical Dosage Forms: Tablets, vol. 1, 2 ed., Lieberman et al. ed., Marcel Dekker, Inc. pub. (1989), pp. 195-246. The enteric coating is suitably applied using any standard coating technique, such as the techniques described in Chapter 5 (“Compression-Coated and Layer Tablets”, by William C. Gunsel et al.), of the same volume.

The present invention is also directed to a method of making the dosage forms of the present invention. In the preferred method, each of the intragranular ingredients is preferably screened or provided in pre-screened form before being dry mixed. If the intragranular ingredients have flow characteristics that make it impracticable to feed the ingredients directly into a tablet press, the ingredients can be granulated prior to compression, for example, by being run through a roller compactor to achieve a suitable ribbon.

When microcrystalline cellulose is included as an excipient in the tablet core, it is preferably included as both an intragranular and as an extragranular ingredient, and added to the other intragranular and extragranular ingredients after each set of ingredients has been mixed, separately. The microcrystalline cellulose is preferably provided pre-screened for particle size prior to addition to the other ingredients. Microcrystalline Cellulose NF Med Powder is an example of one such suitable pre-screened microcrystalline cellulose powder suitable for use in the tablet cores of the present invention.

Once the intragranular ingredients are mixed with all the extragranular ingredients, a compressed tablet is produced therefrom, using any suitable tablet press. Any standard tablet press that does not compress the tablet so far as to damage the water soluble matrix or so compress the tablet that water cannot enter the matrix and solubilize the drug contained therein. The compressed tablets are then completely coated with the enteric coating, comprising an enteric polymer and a pore-former, using any standard coating technique. The enteric coating is preferably applied in the form of a thin layer, causing no more than about an 10% weight gain, more preferably no more than about an 8% weight gain, even more preferably no more than about a 6 % weight gain.

In another embodiment, the present invention is directed to a method of treating or preventing a condition by oral administration of a dosage form of the present invention to a subject. The subject is preferably a mammal, more preferably a mammal selected from the group consisting of a cat, a dog, and a human being. Even more preferably, the subject is a human being. The exact type of dosage form administered to a given subject depends upon the condition to be treated or prevented by the dosage form. For example, when the subject is infected with or in danger of being infected with one or more strains of bacteria, at least one drug of the dosage form is an antibiotic. The dosage form could also suitably include more than one drug, such as an antibiotic and an anti-pain medication. When the subject is infected with or in danger of being infected with a gram positive bacteria, the antibiotic is preferably one, such as clindamycin, that is known to be effective against gram positive bacteria.

The present invention is further illustrated by the following examples. These examples are intended to be illustrative of the invention and should not be used to limit or restrict its scope.

### EXAMPLES

The following examples illustrate one or more of the embodiments of the invention described above.

### Example 1

Various batches of tablets were prepared according to the following procedure, using formulations set forth in Examples 1 and 2 below. In some cases, uncoated controls were prepared as described below, by eliminating step 16, a coating step.

1. All intragranular ingredients except magnesium stearate were weighed.

2. The same ingredients from step 1 were sized through a suitably sized mesh hand screen.

3. The same ingredients were then dry mixed in a suitable blender (a PK blender (Patterson Kelley), in this case) for 7 minutes.

4. The intragranular portion of magnesium stearate (screened through a 30 mesh screen) was weighed and manually blended with a portion of the mixture from step 3, above.

5. The manually blended mixture from step 4 was then combined in a blender with the remainder of the mixture from step 3, and mixed for an additional 3 minutes.

6. The intragranular mixture resulting from step 5 was then run through a roller compactor to achieve a suitable ribbon. Initial granulation was performed by an Alexanderwerk.

7. The material from the first granulation step was separated by sieving using the appropriate mesh screens. Material that meets the predetermined particle size specification was collected. A 20/100 mesh cut was collected (material that passed through a 20 mesh, but was retained on the 100 mesh.

8. The overs from step 7 were milled again using a suitable mill (e.g., a Fitzmill (The Fitzpatrick Company)

9. Steps 6-8 were repeated three times, or until an acceptable yield was obtained.

10. Material retained on the appropriate screen was retained for further processing.

11. All extragranular ingredients except microcrystalline cellulose were weighed. The weight was adjusted to match the yield of material obtained in step 10.

12. The extragranular ingredients weighed in step 11 were dry mixed with the milled intracellular ingredients in a suitable blender (e.g., a PK blender) for 7 minutes.

13. The extragranular magnesium stearate (screened through a 30 mesh screen) was weighed and manually blended with a portion of the mixture of step 12.

14. The premixed ingredients from step 13 were combined with the mixture from step 12 and mixed for an additional 3 minutes.

15. Samples of the resulting mixture from step 14 were compressed into tablets, using a 0.7446 by 0.378 inch modified capsule shaped tooling to obtain tablets of suitable hardness.

16. Finally, the tablets were coated using an 87 to 13 mix of Sureteric/HPMC to a achieve a 4% theoretical weight gain.

### Example 2

Buffered tablets were produced as described in Example 1, according to Formulations 1 through 4, set forth in Table 1, below. The buffer in each tablet was present in the intragranular material. Tablets of Formulation 4 were not coated.

TABLE 1
Buffered Tablet Formulations

| Amount per dosage unit (mg/tablet) | | | | |
|---|---|---|---|---|
| Formula 1 | Formula 2 | Formula 3 | Formula 4 | Material/EDP |

Intragranular Components

| | | | | |
|---|---|---|---|---|
| 600 | 600 | 600 | 600 | Crystalline Clindamycin |
| | | | | Free Base |
| 18.52 | 28.2 | 20.1 | 20.1 | HPMC K4M |
| 138.9 | 141.2 | 200.8 | 200.8 | HPMC K100LV |
| 44 | 44 | 44.35 | 44.35 | Microcrystalline |
| | | | | Cellulose NF |
| | | | | Med Powder |
| 2.31 | 2.35 | 2.5 | 2.5 | Magnesium Stearate NF |
| | | | | Powder Food |
| | | | | Grade-V-Bolted |

Extragranular Components

| | | | | |
|---|---|---|---|---|
| 119.86* | *122.90 | 133.75* | 133.75* | Microcrystalline |
| | | | | Cellulose NF |
| | | | | Med Powder |
| 2.31 | 2.35 | 2.5 | 2.5 | Magnesium Stearate NF |
| | | | | Powder Food |
| | | | | Grade-V-Bolted |
| 925.9 | 941 | 1004 | 1004 | Tablet total |

Coating Materials

| | | | | |
|---|---|---|---|---|
| 4.81 | 4.89 | 5.22 | None | HPMC |
| 32.19 | 32.75 | 34.94 | None | Sureteric (EDP#NA) |
| 962.9 | 978.64 | 1044.16 | 1004 | Total system weight |

*Weight adjusted for potency of Free Base.

Each of Formulae 1 through 4 was tested in vitro dissolution results were collected at 1, 2, 3, 4, 6, 8, 12, 16, 20, and 24 hours after time zero.

The three coated tablets all had similar release rate profiles for the first two hours in the low pH conditions. The release rates of tablets of Formula 2 and of Formula 3 remained relatively constant for at least 8 hours, before slowing down and beginning to level off. The tablets of Formula 1 had a similar release rate profile, except that the release rate increased slightly after the first two hours. The uncoated tablets (Formula 4) had a significantly higher release rate within the first two hours of administration, and the release rate slowed considerably after that point, under high pH conditions. In other words, release of drug from the uncoated tablets was found to be pH dependent, whereas all of the coated tablets tested in this assay released clindamycin at a pH independent release rate.

In other tests, it was noted that when buffer was included in the formulations, the buffer, took up a relatively large amount of space in each tablet, limiting the amount of drug that could be accommodated without increasing tablet size. Therefore, it was decided to produce dosage forms of coated tablets in the same way as described above, without any buffer being included in the tablet, to see if the unbuffered tablets would be have pH independent drug release profiles, as did the buffered tablets.

### Example 3

In this example, buffered and unbuffered, coated and uncoated tablets were produced and tested in vitro to determine whether pH independent tablets could be produced without a buffer in the tablet core. One formulation was also produced and tested with an enteric coating, without a pore former present therein. Specifically, tablets were produced as described in Example 1 minus the extragranular incorporation steps, with modifications made to the procedure set forth therein to produce uncoated tablets (Formulas 5, 7, and 9), to include a buffer in the tablet core of certain tablets (Formulas 8 and 9), and to produce tablets coated with an enteric coating without a pore former (Formula 6), as set forth in Table 2, below. As in the tablets of Example 2, when a buffer was present in the tablets produced in this Example, it was present in the intragranular material.

TABLE 2
Coated/Uncoated, Buffered/Unbuffered Tablet Formulations

| Amount per dosage unit (mg/tablet) | | | | | |
|---|---|---|---|---|---|
| Formula | Formula | Formula | Formula | Formula | |
| 5 | 6 | 7 | 8 | 9 | Material/EDP |

Intragranular Components

| | | | | | |
|---|---|---|---|---|---|
| 600 | 600 | 600 | 600 | 600 | Crystalline Clindamycin |
| | | | | | Free Base |
| 20 | 20 | 20 | 24.1 | 120.5 | HPMC K4M |
| 200.7 | 200.7 | 281 | 337.3 | 241 | HPMC K100LV |
| 180 | 180 | 100 | 120.5 | 120.5 | Microcrystalline |
| | | | | | Cellulose NF Med |
| | | | | | Powder |
| 2.61 | 2.61 | 2.61 | 3.13 | 3.13 | Magnesium Stearate NF |
| | | | | | Powder Food Grade-V- |
| | | | | | Bolted |
| None | None | None | 120.5 | 120.5 | Potassium Phosphate |
| | | | | | Monobasic |
| 1004 | 1003 | 1004 | 1205 | 1205 | Tablet total |

Coating Materials

| | | | | | |
|---|---|---|---|---|---|
| none | none | none | 9.64 | none | HPMC |
| none | 40.16 | none | 38.56 | none | Sureteric (EDP#NA) |
| | 1043.5 | 1004 | 1253.2 | 1205 | Total system weight |

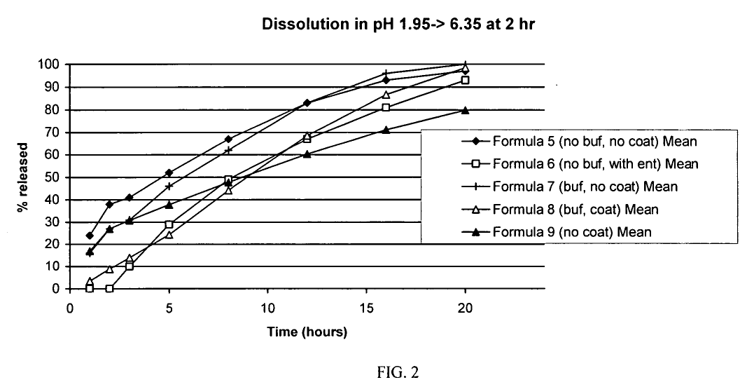

Each of the formulations described above was tested by being placed in a container with an aqueous solution of a pH of 1.95 for two hours. At the end of the two hours, the pH of the solution was raised to pH 6.35. Aliquots of the aqueous solution 16, 20, and 24 hours after introduction of the tablet thereto. The percent of crystalline clindamycin free base released into the solution at each time point was determined by a high pressure liquid chromatography (“HPLC”) assay. The results of this assay are presented in FIG. 2.

FIG. 2 shows that all of the uncoated formulations released clindamycin at a significantly faster rate at the lower pH than they did at the higher pH. Both the tablets of unbuffered and uncoated Formulation 5, and tablets of unbuffered and uncoated Formulation 7 had released over 80% of the clindamycin by the 12 hour time point, and had released about 95% of the clindamycin present therein by the 16 hour time point. The tablets from Buffered uncoated Formulation 9 also displayed a significant pH independence on release rate. This Formulation had a much slower release rate at the higher pH level due to the use of additional polymer in the formulation.

The tablets coated with an enteric coating (Formula 6) did not begin to release clindamycin until after the 2 hour time point, when the pH of the solution was raised from pH 1.95 to pH 6.35. Thus, the purely enteric coated tablets were also found to have a pH dependent release rate.

In contrast to all of the other tablet formulations tested above, the tablets of Formula 8, with an enteric coating containing a pore former (i.e., HPMC), released clindamycin at a substantially linear rate, through the change in pH at the two hour time point, and continued to release clindamycin until about the 20 hour time point. At the 12 hour time point, less than 70% of the clindamycin of Formula 8 had been released; and at the 16 hour time point, only about 85% of the clindamycin had been released.

### Example 4

Four dosage forms (Formulations 10-13) of unbuffered tablets of crystalline clindamycin free base were prepared according to the procedure of Example 1, using the formulae (Formulations 10-13), described in Table 3, below.

TABLE 3
Unbuffered Tablets

| Amount per dosage unit (mg/tablet) | | | | |
|---|---|---|---|---|
| Formula | Formula | Formula | Formula | |
| 10 | 11 | 12 | 13 | Material/EDP |

Intragranular Components

| | | | | |
|---|---|---|---|---|
| 626.54* | 626.54* | 626.54* | 626.54* | Crystalline Clindamycin |
| | | | | Free Base |
| 20.1 | 20.1 | 18.54 | 28.2 | HPMC K4M |
| 200.8 | 200.8 | 138.9 | 141.2 | HPMC K100LV |
| 44.35 | 44.35 | 44.0 | 44.0 | Microcrystalline Cellulose NF |
| | | | | Med Powder |
| 2.5 | 2.5 | 2.31 | 2.35 | Magnesium Stearate NF |
| | | | | Powder Food Grade-V-Bolted |

Extragranular Components

| | | | | |
|---|---|---|---|---|
| 107.21* | 107.21* | 93.32 | 96.36 | Microcrystalline Cellulose NF |
| | | | | Med Powder |
| 2.5 | 2.5 | 2.31 | 2.35 | Magnesium Stearate NF |
| | | | | Powder Food Grade-V-Bolted |
| 1004 | 1004 | 925.9 | 941 | Tablet total |

Coating Materials

| | | | | |
|---|---|---|---|---|
| none | 5.22 | 4.81 | 4.89 | HPMC |
| none | 34.94 | 32.19 | 32.75 | Sureteric (EDP#NA) |
| 1004 | 1044.16 | 962.9 | 978.64 | Total system weight |

*Weight adjusted for potency of Free Base.

Tablets of Formulae 10 through 13 were tested for release rate profiles in vitro, in the same way described in Example 3, above. Tablets of formulae 11 through 13, the coated tablets, produced pH independent release rate profiles, while tablets of Formula 10 were clearly pH dependent. Surprisingly, it was found that one could achieve pH independent release from an unbuffered coated tablet produced as described above.

### Example 5

After testing a number of different unbuffered coated tablet dosage forms in experiments such as that described in Example 4, above, one particularly stable dosage form (Formulation 15) with a pH independent release profile was identified. This dosage form, described in Table 4, below, was produced according to the procedure of Example 1, above. An uncoated version of the same dosage form, Formula 14, was produced according to the same procedure, by omitting the coating step.

Coated Unbuffered Tablet Formulation

| Formula | Formula | |
|---|---|---|
| 14 | 15 | Material/EDP |

Intragranular Components

| | | |
|---|---|---|
| 600* | 600* | Crystalline Clindamycin Free Base |
| 18.52 | 18.52 | HPMC K4M |
| 138.9 | 138.9 | HPMC K100LV |
| 44 | 44 | Microcrystalline Cellulose NF Med Powder |
| 2.31 | 2.31 | Magnesium Stearate NF Powder Food Grade-V- |
| | | Bolted |

Extragranular Components

| | | |
|---|---|---|
| 119.86* | 119.86* | Microcrystalline Cellulose NF Med Powder |
| 2.31 | 2.31 | Magnesium Stearate NF Powder Food Grade-V- |
| | | Bolted |
| 925.9 | 925.9 | Tablet total |

Coating Material

| | | |
|---|---|---|
| none | 4.81 | HPMC |
| none | 32.19 | Sureteric (EDP#NA) |
| | 247.6 mg | (Purified Water USP) |
| | 962.9 | Total system weight |

*Weight adjusted for potency of Free Base.

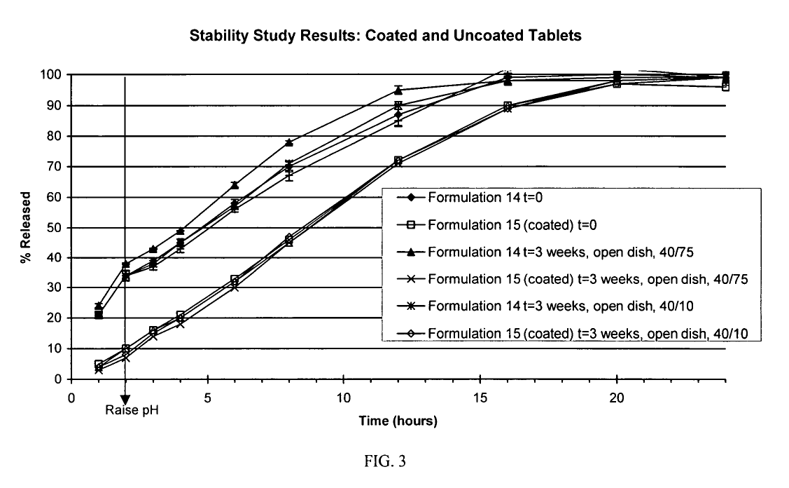

The release rate profiles of tablets of Formulae 14 and 15 were tested, after the tablets were stored under a variety of different conditions. The resulting release rate profiles are illustrated in FIG. 3. One set of tablets of each formula was tested at time point zero, without any storage. A second set of tablets was tested after storage for three weeks in an open dish, at 40° C. and 75% humidity. A third set of tablets was tested after storage for three weeks in an open dish at 40° C. and 10% humidity. A fourth set of tablets was tested after storage for three weeks in a closed container with a desiccant, at 40° C. and 75% humidity. The tablets were placed in a solution having a pH of 1.95 at time zero. The pH of the solution was maintained at 1.95 until two hours after time zero, at which time it was raised to pH 6.35. The amount of crystalline clindamycin free base released from each tablet was measured at time zero, and at 1, 2, 3, 4, 6, 8, 12, 16, 20, and 24 hours after time zero.

All of the tablets of Formula 15 stored under the various different conditions described above produced essentially identical pH independent drug release profiles, releasing crystalline clindamycin free base at a linear release rate that continued through 12 hours after time zero. At the 16 hour time point, 98% to 100% of the clindamycin had been released from the uncoated tablets, while about 90% of the clindamycin had been released from the coated tablets.

The uncoated tablets (of Formula 14) also all had pH dependent drug release profiles that varied from one another, with the tablets stored for three weeks in an open dish at 40° C. and 75% humidity having the fastest release rate of the samples tested, and humidity conditions having the slowest and most constant release rate once the pH was lowered.

### Example 6

Additional dosage forms were made according to steps 1-9 and 15 of the procedure of Example 1 (i.e. eliminating mixing of extragranular with intragranular components and a coating step), using a modified form of Formula 15, wherein HPMC K4M was the only intragranular or extragranular polymer component. Formulae produced and tested in this Example are described in Table 4, below.

TABLE 4
K4M Formulations

| Amount per dosage unit (mg/tablet) | | | | | |
|---|---|---|---|---|---|
| Formula | Formula | Formula | Formula | Formula | |
| 16 | 17 | 18 | 19 | 20 | |
| (10%) | (12%) | (14%) | (15%) | (19%) | Material/EDP |

Intragranular Components

| | | | | | |
|---|---|---|---|---|---|
| 600 | 600 | 600 | 600 | 600 | Crystalline |
| | | | | | Clindamycin Free Base |
| 79.4 | 97.84 | 117.3 | 127.5 | 162.2 | HPMC K4M |
| 112.3 | 115.37 | 118.6 | 120.3 | 84.99 | Microcrystalline |
| | | | | | Cellulose NF Med |
| | | | | | Powder |
| 2.1 | 2.12 | 2.18 | 2.21 | 2.21 | Magnesium Stearate NF |
| | | | | | Powder Food Grade-V- |
| | | | | | Bolted |
| 793.7 | 815.35 | 838.1 | 849.9 | 849.5 | Tablet total |

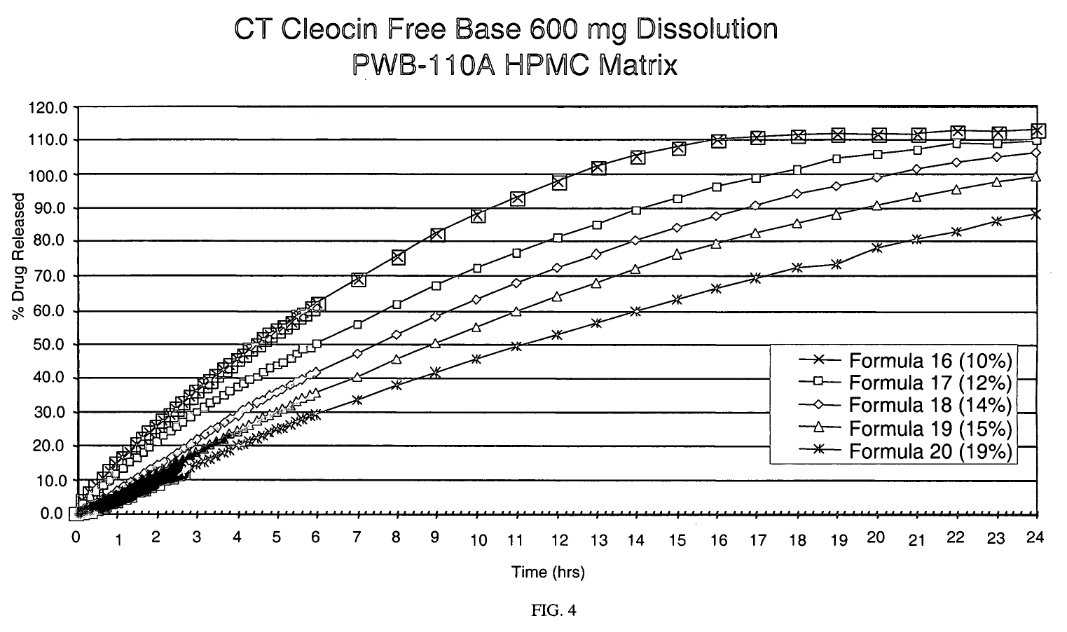

Tablets of Formulae 16 through 20 were tested in vitro for release rate profiles at various time points in a potassium phosphate buffer, pH 6.8. The results of this assay are illustrated in FIG. 4. All five formulations exhibited extended release of crystalline clindamycin free base. The release rates decreased with increasing percent by weight HPMC K4M in each formulation, with tablets of Formula 20 having the lowest and most extended release rate of all the formulae tested in this example.

### Example 7

Additional dosage forms were made according to steps 1-9, 15, and 16 of the procedure of Example 1 (i.e. Including a coating step; but, eliminating mixing of extragranular with intragranular components), using a modified form of Formula 15, wherein NaCMC was used instead of HPMC as an intragranular polymer component. Formulae produced and tested in this Example are described in Table 5, below.

TABLE 5
NaCMC based Formulations

| Amount per dosage | | | |
|---|---|---|---|
| unit (mg/tablet) | | | |
| Formula | Formula | Formula | |
| 21 | 22 | 23 | Material/EDP |

Intragranular Components

| | | | |
|---|---|---|---|
| 600 | 600 | 600 | PNU-21251 Clindamycin Free Base |
| 56.1 | 69.6 | 83.6 | Na CMC 7H3SXF PH |
| 144.4 | 147.3 | 150.4 | Microcrystalline Cellulose NF |
| | | | Med Powder |
| 2.1 | 2.12 | 2.17 | Magnesium Stearate NF Powder Food |
| | | | Grade-V-Bolted |
| 802 | 819 | 836 | Tablet total |

Coating Materials

| | | | |
|---|---|---|---|
| 4.17 | 4.26 | 4.35 | HPMC |
| 27.9 | 28.5 | 29.1 | Sureteric (EDP#NA) |
| 834 | 851.8 | 869.44 | Total system weight |

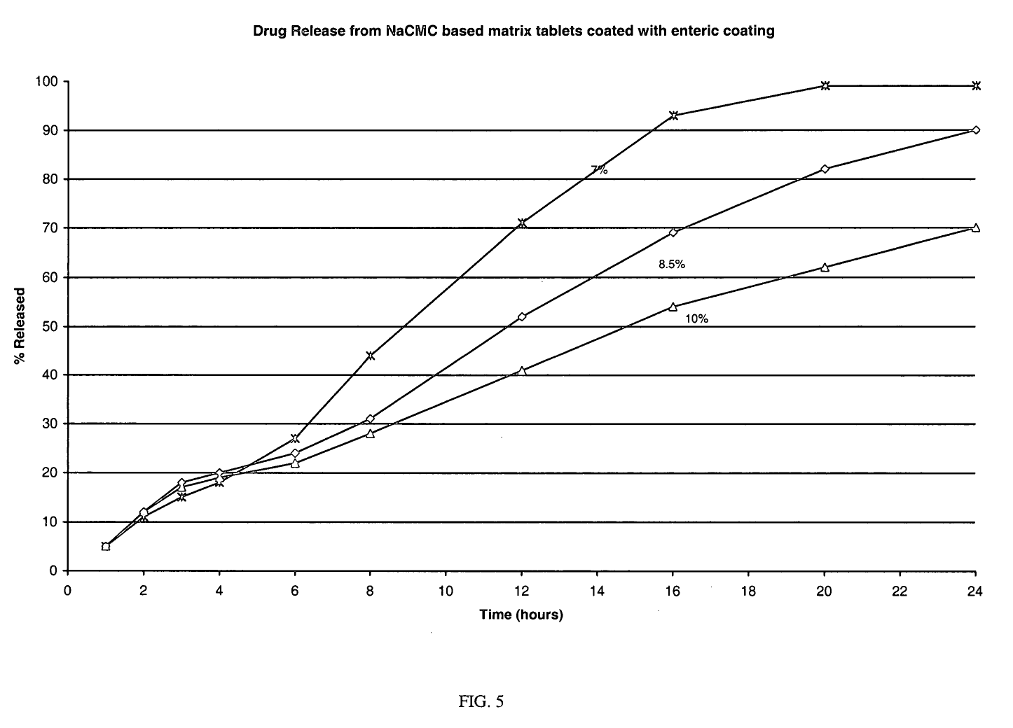

Tablets of Formulae 21 through 23 were tested in vitro for release rate profiles at various time points, as described in Example 3, above. The results are illustrated in FIG. 5, with the data for Formula 21 (wherein about 7% by weight of the core was NaCMC) plotted with “*” symbols, with the data for Formula 22 (wherein about 8.5% by weight of the core was NaCMC) plotted with “♦” symbols, and with the data for Formula 23 (wherein about 10% by weight of the core was NaCMC) plotted with “▴” symbols.

All three formulations exhibited pH independent release rates. Of the three formulae tested, Formula 21, the formula with the smallest weight percent of NaCMC, had the most rapid release rate. By the 16 hour time point, over 90% of the clindamycin in that formulation had been released into solution. By the same time point, only about 70% of the clindamycin in Formula 22 and about 54% of the clindamycin in Formula 21 had been released.

We claim:

1. An extended release dosage form comprising:
a tablet core comprising at least one drug and a water soluble polymer in the form of a matrix, the tablet core being surrounded by an outer surface, and
an enteric coating completely covering the outer surface of the tablet core, the coating comprising an enteric polymer and a pore-former distributed within the enteric polymer.

2. The dosage form of claim 1, wherein the enteric coating reduces any burst effect that would be exhibited by the tablet core in the absence of the enteric coating.

3. The dosage form of claim 1, wherein the drug is less soluble in an aqueous solution at a pH of greater than about pH 5.0 than it is at a pH of below about pH 5.0.

4. The dosage form of claim 3, wherein the drug is an antibiotic.

5. The dosage form of claim 4, wherein the antibiotic is clindamycin.

6. The dosage form of claim 5, wherein the clindamycin is in the form of crystalline clindamycin free base.

7. The dosage form of claim 1, wherein the water soluble polymer is selected from the group consisting of: a cellulose ether, hydroxypropylcellulose, sodium carboxymethyl cellulose, xanthan gum, acacia, tragacanth gum, guar gum, karaya gum, alginates, gelatin, albumin.

8. The dosage form of claim 1, wherein the water soluble polymer is hydroxypropylmethyl cellulose.

9. The dosage form of claim 1, wherein the tablet core further comprises at least one binder.

10. The dosage form of claim 9, wherein the at least one binder is microcrystalline cellulose and magnesium stearate.

11. The dosage form of claim 1, wherein the enteric polymer is selected from the group consisting of: a methacrylic acid/methacrylic acid ester copolymer, a methacrylic acid/acrylic acid ester copolymer, cellulose acetate phthalate, hydroxypropyl methylcellulose pthalate, hydroxypropyl methyl cellulose acetate succinate, cellulose acetate trimellitate, and polyvinyl acetate phthalate.

12. The dosage form of claim 1, wherein the enteric polymer is a polyvinyl acetate phthalate.

13. The dosage form of claim 1, wherein the pore-former is a water soluble polymer.

14. The dosage form of claim 13, wherein the water soluble polymer pore-former is hydroxypropylmethyl cellulose.

15. A pH independent extended release dosage form comprising:
a tablet core matrix, comprising a crystalline clindamycin free base and a water soluble polymer in the form of a matrix, the tablet core having an outer surface, and
an enteric coating covering the entire outer surface of the tablet core, comprising an enteric polymer and a pore-former distributed within the enteric polymer.

16. The dosage form of claim 15, wherein the water soluble polymer is selected from the group consisting of: a cellulose ether, hydroxypropylcellulose, sodium carboxymethyl cellulose, xanthan gum, acacia, tragacanth gum, guar gum, karaya gum, alginates, gelatin, albumin.

17. The dosage form of claim 15, wherein the water soluble polymer is hydroxypropylmethyl cellulose.

18. The dosage form of claim 15, wherein the tablet core further comprises at least one binder.

19. The dosage form of claim 18, wherein the at least one binder is microcrystalline cellulose and magnesium stearate.

20. The dosage form of claim 15, wherein the enteric polymer is selected from the group consisting of: a methacrylic acid/methacrylic acid ester copolymer, a methacrylic acid/acrylic acid ester copolymer, cellulose acetate phthalate, hydroxypropyl methylcellulose pthalate, hydroxypropyl methyl cellulose acetate succinate, cellulose acetate trimellitate, and polyvinyl acetate phthalate.

21. The dosage form of claim 15, wherein the enteric polymer is polyvinyl acetate phthalate.

22. A method of treating or preventing a gram positive bacterial infection in a subject, comprising: orally administering to the subject a tablet of the dosage form of claim 15, wherein the tablet core comprises a pharmaceutically effective amount of the crystalline clindamycin free base.

23. The method of claim 22, wherein the subject is a mammal.

24. The method of claim 22, wherein the subject is a human.

25. The method of claim 22, wherein the amount of the crystalline clindamycin free base in the tablet core is 500-2000 mg.

26. A method of making a drug release dosage form, comprising the steps of:
a. dry mixing intragranular ingredients comprising a drug, hydroxypropylmethyl cellulose, microcrystalline cellulose, and magnesium stearate, thereby producing an intragranular mixture;
b. processing the intragranular mixture through a roller compactor;
c. dry mixing extragranular ingredients, comprising hydroxypropylmethyl cellulose, microcrystalline cellulose, and magnesium stearate with the intragranular ingredients, thereby producing a core tablet mixture;
d. pressing the core tablet mixture in a tablet press to produce a tablet core;
e. coating the tablet core with an enteric coat comprising an enteric polymer and a pore former.

27. The method of claim 26, wherein at least one of the intragranular ingredients is provided in a sized form for dry mixing in step (a).

28. The method of claim 26, wherein all intragranular ingredients except the magnesium stearate are dry mixed in a first mixing step, producing an intermediate intragranular mixture, followed by dry mixing the intermediate intragranular mixture with the magnesium stearate.

29. The method of claim 26, wherein all extragranular ingredients are initially mixed together with the intragranular ingredients mixture from step (a) except for magnesium stearate, and magnesium stearate is subsequently mixed therewith.

30. The method of claim 26, wherein the drug is an antibiotic.

31. The method of claim 30, wherein the drug is crystalline clindamycin free base.
